(19) United States
(12) Patent Application Publication
(10) Pub. No.: US 2026/0125668 A1
(43) Pub. Date: May 7, 2026

(54) DEVICES, SYSTEMS, AND METHODS RELATING TO MICROFLUIDIC PURIFICATION OF NUCLEIC ACIDS

(71) Applicant: University of Florida Research Foundation, Inc., Gainesville, FL (US)

(72) Inventors: Jason Edward BUTLER, Gainesville, FL (US); Anthony J. LADD, Gainesville, FL (US); Jiayi WANG, Gainesville, FL (US)

(21) Appl. No.: 19/376,112

(22) Filed: Oct. 31, 2025

Related U.S. Application Data
(60) Provisional application No. 63/715,486, filed on Nov. 1, 2024.
(60) Provisional application No. 63/743,442, filed on Jan. 9, 2025.
(60) Provisional application No. 63/807,946, filed on May 19, 2025.

(51) Int. Cl. C12N 15/10; B01L 3/00

(52) U.S. Cl. CPC C12N 15/1003; B01L 3/502715; B01L 3/502761; B01L 2300/047; B01L 2300/0645; B01L 2300/16; B01L 2400/0457; B01L 2400/0487

(57) ABSTRACT

Described herein are devices, systems, and methods related to isolation and/or detection and/or trapping of nucleic acids utilizing microfluidics. In an embodiment, described herein is a microfluidic trap. In embodiments, a microfluidic trap can comprise an inlet region having an injection port, a first extraction port, and a first electrode; an outlet region having an outlet port and a second electrode; and a microfluidic channel providing fluidic communication between the inlet region and outlet region. In certain aspects, nucleic acids that are trapped comprise genomic DNA.

### CROSS-REFERENCE TO RELATED APPLICATION[S]

This application claims priority to, and the benefit of, U.S. Provisional Patent Application Ser. No. 63/715,486, entitled “MICROFLUIDIC DNA PURIFICATION PROCESS” and filed on Nov. 1, 2024, the entire contents of which are incorporated herein by reference as if set forth in its entirety.

This application claims priority to, and the benefit of, U.S. Provisional Patent Application Ser. No. 63/743,442, entitled “MICROFLUIDIC DNA PURIFICATION PROCESS” and filed on Jan. 9, 2025, the entire contents of which are incorporated herein by reference as if set forth in its entirety.

This application claims priority to, and the benefit of, U.S. Provisional Patent Application Ser. No. 63/807,946, entitled “DEVICES, SYSTEMS, AND METHODS RELATING TO MICROFLUIDIC PURIFICATION OF NUCLEIC ACIDS” and filed on May 19, 2025, the entire contents of which are incorporated herein by reference as if set forth in its entirety.

### STATEMENT REGARDING FEDERALLY SPONSORED RESEARCH OR DEVELOPMENT

This invention was made with government support under Grant No(s) 1804302 & 2222688, awarded by the National Science Foundation. The government has certain rights in the invention.

### SEQUENCE LISTING

The instant application contains a Sequence Listing which has been submitted electronically in .XML format and is hereby incorporated by reference in its entirety. Said. XML copy, created on Oct. 29, 2025, is named “UFPT19570US004_222112_1970_sequence listing.xml” and is 2,932 bytes in size.

### BACKGROUND

Microfluidic processing and analysis of DNA continues to be a vibrant area of research and development due to the reduced sample sizes and possibilities for integrating sample preparation and analysis into a single fully automated device. A cell lysate contains a number of contaminants that must be removed before any analysis of the DNA is possible. These include components native to the biological sample, such as polysaccharides in plant-based samples, or reagents used in the lysing process, such as salts and detergents. In practice, purification is typically performed on relatively large samples (milliliter volumes) using liquid or solid-phase extraction methods, including magnetic beads that bind reversibly to the DNA. However, these methods are time-consuming and labor-intensive, involving multiple pipette transfers and centrifugation steps. The stresses created by centrifuging fragment individual DNA strands, which is undesirable when preparing samples for long-read sequencing. There is a need to address the aforementioned deficiencies and inadequacies accordingly.

### SUMMARY

Described herein are devices, kits, systems, compositions, and methods related to isolation and/or detection and/or trapping of nucleic acids utilizing fluid flow and electric field application within microfluidics.

Described herein are microfluidic traps, systems, and methods of use. In embodiments, described herein is a microfluidic trap, comprising an inlet region having an injection port, a first extraction port, and a first electrode; an outlet region having an outlet port and a second electrode; and a microfluidic channel providing fluidic communication between the inlet region and outlet region. In certain aspects, there may be electrodes in the microfluidic device and outside of the microfluidic device. The polyelectrolyte trapping part of the process can be driven by the external electrodes, but can also be driven by the internal electrodes. The internal electrodes must be used for DNA collection in order to avoid reintroducing contaminants.

In embodiments, the first electrode and the first extraction port are in close proximity and separated from the injection port by an inlet distance. In embodiments, the second electrode and the outlet port are separated by an outlet distance. In embodiments, the injection port, extraction port, and outlet port are in line with the longitudinal axis of the microfluidic channel. In embodiments, the inlet region further comprises one or more reservoirs adjacent to the longitudinal axis of the microfluidic channel, wherein the first electrode is within one of the one or more reservoirs and the second electrode is in line with the longitudinal axis of the microfluidic channel. In certain aspects, the first electrode can be positioned in a secondary, side channel or area which is connected to the inlet, or in line with the inlet. In certain aspects, an inlet electrode would need to be near to the extraction port. In certain aspects, the second electrode probably is inline, or at least not offset very far, from the inlet or microfluidic channel. In embodiments, the first electrode and the second electrode comprise a noble metal, one or more metal alloys, or a combination of any thereof. In embodiments, the first electrode and second electrode can be coated with a charge neutral polymer having a sufficient pore size to prevent passage of genomic DNA or other nucleic acids through the pores. In embodiments, microfluidic channel can be about 1 mm to about 100 cm long. In embodiments, the microfluidic channel, separation channel, or both are shorter than the entire length of the device. In embodiments, the inlet distance is about 0.5 cm to about 5 cm. In embodiments, the outlet distance is about 0.5 cm to about 5 cm. In embodiments, the inlet region, outlet region, microfluidic channel, or any combination of any thereof are laser etched in acrylic or glass, silicon, polydimethylsiloxane (PDMS), paper, a thermoplastic material, or a combination of any thereof. In embodiments, the acrylic is bonded multilayer acrylic. In embodiments, the inlet region, outlet region, microfluidic channel, or any combination of any thereof comprise glass, silicon, polydimethylsiloxane (PDMS), paper, a thermoplastic material, or a combination of any thereof. In embodiments, the thermoplastic material is polycarbonate. In embodiments, the inlet region further comprises an inlet flow adaptor. In embodiments, the outlet region further comprises an outlet flow adaptor. In embodiments, the inlet flow adaptor and outlet flow adaptor are each male or female luer connectors.

Described herein are systems comprising any microfluidic trap according to the present disclosure. In embodiments, a system comprises a microfluidic trap as described herein, a voltage generator, one or more fluid flow valves, and a fluid flow generator. In embodiments, systems further comprise a fluid source in fluidic communication with the inlet region of the microfluidic trap. In embodiments, systems further comprise a fluid collection device in fluidic communication with the outlet region of the microfluidic trap. In embodiments, systems further comprise a flow valve in fluidic communication with the fluid source. In embodiments, the fluid flow generator is an active or passive fluid flow generator. In embodiments, the active fluid flow generator is a fluidic pump. In embodiments, the passive fluid flow generator is gravitational flow created by a difference in height between the fluid source and the fluid collection device. In embodiments for gravity flow, the only requirement is that the source is higher than the collection device.

Described herein are methods of using microfluidic traps and systems according to the present disclosure. In embodiments, described herein is a method, comprising establishing a fluid flow in a system as described herein; providing a first electric field across the longitudinal axis of the microfluid channel with the voltage generator; injecting a biological sample comprising genomic DNA into the injection port of the system; waiting a first period of time; stopping the fluid flow; removing the first electric field; providing a second electric field across the first and second electrodes of the microfluidic trap with the voltage generator; waiting a second period of time; and collecting the remaining nucleic acids from the extraction port of the microfluidic trap. In embodiments, the biological sample comprises cell lysate comprising genomic DNA. In embodiments, the flow has a flow rate of about 1 to about 500 microliters per minute. In embodiments, the first electric field has a strength of about 0 to about 500 V/cm. In embodiments, the polarity of the first electric field is such that the polarity attracts a polyelectrolyte or nucleotide to the inlet region, for example, to account for external electrodes operating the first electric field.

In embodiments, the first period of time is about 1 minute to about 5 hours. In embodiments, the second electric field has a strength of up to about 500 V/cm. In embodiments, the polarity of the second electric field is such that the polarity attracts a polyelectrolyte or nucleotide to the inlet region. In embodiments, the second period of time is about 1 minute to about 5 hours. In embodiments, the remaining nucleic acids have a size of greater than about 5 kb. In embodiments, the polarity of the second electric field is such that it attracts the polyelectrolyte/nucleotides of interest to the inlet. The skilled artisan would recognize that field polarity will depend upon the overall charge of the polyelectrolyte of interest.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1C: Device for electrohydrodynamic separation (EHS) of DNA. (FIG. 1A) Scale drawing of the microfluidic device showing the four key regions—inlet, trap, separation channel, outlet—the four ports (A-D), and the on-chip electrodes (E₁ and E₂). An alternative design with a longer separation channel is shown in FIG. 6. (FIG. 1B) Illustration of the separation mechanism in the channel (˜1% of the channel is shown). The average convective flow (blue) is significantly larger than the electrophoretic velocity (red). DNA (green) close to the wall is driven backward by electrophoresis, while contaminants (gray) move forward under the larger convective flow, regardless of their charge state. (FIG. 1C) Sketch of the experimental setup. The height difference driving the flow is measured from an origin established by opening the leveling valve. A voltage difference is applied to electrodes in the buffer reservoirs, with the inlet reservoir at a positive voltage with respect to the outlet reservoir.

FIGS. 2A-2B: DNA trapping at the entry to the separation channel. (FIG. 2A) Fluorescence images of DNA under different combinations of flow (set by ΔH) and electric fields (set by the voltage V). Images were taken after 15 min. (FIG. 2B) DNA concentrations in the region of the trap, after injecting a 10 μL sample (50 ng/μL) of E. coli lysate. The markers represent variations in DNA concentrations with time, under the same combinations of fluid flow and electric field as the images.

FIG. 3: PCR amplification of E. coli DNA. The curves show the concentration (as measured by ΔRₙ) for both labeled (red) and unlabeled (blue) samples, as well as for the unpurified lysate (black). The slope of the dashed line indicates a doubling of the concentration every cycle. A threshold of ΔRₙ=25 is taken, corresponding to the beginning of the exponential growth, to determine the Cq values in Table 1.

FIGS. 4A-4D: TapeStation electropherograms. (FIG. 4A) E. coli lysate (50 ng/μL) on Genomic DNA ScreenTape. (FIG. 4B) Purified E. coli DNA (4 ng/μL) on Genomic DNA ScreenTape. (FIG. 4C) Diluted E. coli lysate (3.5 ng/μL) on High Sensitivity DNA ScreenTape. (FIG. 4D) Purified E. coli DNA (3 ng/μL) on High Sensitivity DNA ScreenTape. Samples in (FIG. 4B) and (FIG. 4D) were extracted using the 80 mm channel (FIG. 6). The Genomic DNA ScreenTape includes a lower marker at 100 bp, and the High Sensitivity ScreenTape has markers at 15 bp and 10 kbp.

FIGS. 5A-5B: Concentration of DNA in an approximately 350 μm×250 μm window positioned at the entrance to the (20 mm) separation channel. Samples of 10 μL, containing a mixture of DNA (15 ng/μL) and BSA (30 mg/mL), were injected into the device. The legend indicates the pressure and voltage differences in each experiment. (FIG. 5A) DNA (blue symbols) and BSA (red symbols) concentrations in the trap as a function of time. The concentrations are averaged over the height of the device (200 μm) and over the viewing window. The left y axis indicates the DNA concentrations (in ng/μL) and the right axis the BSA concentrations (in mg/mL). The Inset figure shows the BSA concentration during the last 15 min of the experiment. (FIG. 4B) DNA and BSA concentrations in extracted samples. The error bars are based on variations between three separate extractions.

FIG. 6: Scale drawing of a microfluidic device with a longer separation channel. The key regions—inlet, trap, separation channel, outlet—the 4 ports (A-D), and the on-chip electrodes (E₁, E₂) are the same as in FIG. 1A.

FIGS. 7A-7C: DNA migration in a channel. (FIG. 7A) The ratio of tranverse to axial velocities of λ-DNA in a shear flow. (FIG. 7B) Mean fluorescence intensity of DNA (orange) and buffer (green) as a function of the distance from the wall. The inset shows a confocal image near the wall showing the distribution of DNA (orange) in the separation channel. (FIG. 7C) Confocal scan showing the distribution of DNA in the inlet region behind the channel. Panels FIG. 7B and FIG. 7C were reproduced from Ref. (4) with permission from the Royal Society of Chemistry.

FIGS. 8A-8B: Mass of trapped A-DNA from varying injected amounts and different channel lengths. (FIG. 8A) Fluorescence intensity of DNA at the outlet was measured after turning off the flow and reversing the electric field. The plots are an average of three experiments. (FIG. 8B) Mass of trapped DNA from the injected samples in panel (FIG. 8A). The error bars reflect the variation in concentration over 3 experiments.

FIG. 9: Fluorescent images of DNA from human cell lysate (HEK 293). The top row shows DNA trapping 10 minutes after lysate injection, while the bottom row shows the flushing of trapped DNA up to 30 seconds after turning off the electric field.

FIGS. 10A1-10B6: Electropherograms from human cells (HEK 293): (FIGS. 10A1-10A2) the lysate (46 ng/μL) on a Femto Pulse System (left) and TapeStation Genomic DNA ScreenTape (right). For Femto Pulse analysis, the lysate was diluted to 500 pg/μL to fit the concentration range (50-500 pg/μL). (FIGS. 10B1-10B6) Three purified human DNA samples (5.1, 1.4, and 4.3 ng/μL) on a Femto Pulse System (left) and TapeStation Genomic DNA ScreenTape (right). For Femto Pulse analysis, the samples were diluted to 250, 150, and 250 pg/μL to fit the concentration range (50-500 pg/μL).

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, microbiology, biochemistry, molecular biology, cellular biology, tissue culture, and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with the micro-organisms in nature. Trapped is another word for isolation in the context of the present disclosure.

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A sample is a composition that contains matter from at least one cell of a subject, for example, nucleic acids. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “biological sample” can refer to a sample comprising cells or cellular debris, for example, nucleic acids (in particular genomic DNA) from any “individual”, “organism”, “host”, “subject”, or “patient” described herein. The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples. In embodiments, the biological sample comprises urine; a cervical or vaginal swab; or an endotracheal aspirate. Samples comprise nucleic acids according to the present disclosure.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The terms “administering,” “delivering,” and “introducing,” can be used interchangeably to indicate the introduction of a biological sample into devices and systems as described herein. The biological sample can be administered through any appropriate means that results in the delivery of at least a portion of the biological sample to a desired location in any device or system described herein.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-87 (2013); Xie et al. “Adding amino acids to the genetic repertoire,” Curr. Opin. Chem. Biol. 9 (6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-10 and Altschul et al. (1977) Nucleic Acids Res. 25:3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

Those skilled in the art will appreciate that the term “composition”, as used herein, can be used to refer to a discrete physical entity that comprises one or more specified components. The term “composition” as used herein refers to a product comprising any one or more of the specified ingredients disclosed herein in any one of more of the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier. In general, unless otherwise specified, a composition can be of any suitable form—e.g., gel, liquid, solid, etc.

### Discussion

Described herein are devices, systems, and methods related to isolation and/or detection and/or trapping of nucleic acids (and other non-nucleic acid polyelectrolytes) utilizing microfluidics. Improvements over prior technology include the incorporation of one or more electrodes and one or more electric fields to improve the purity of isolated nucleic acids. Additionally, described herein is the isolation and/or trapping of nucleic acids from bacterial samples or samples comprising mammalian DNA.

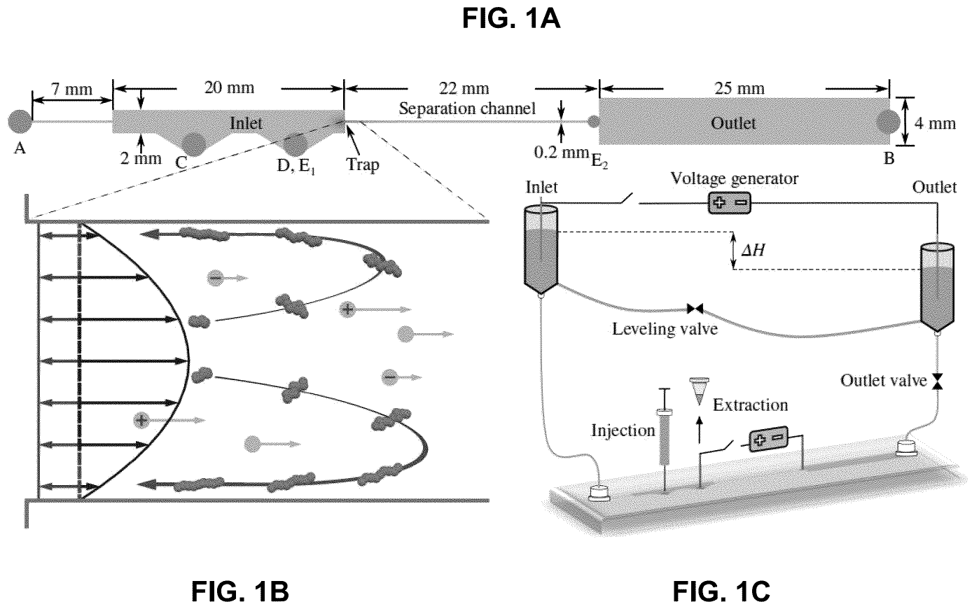

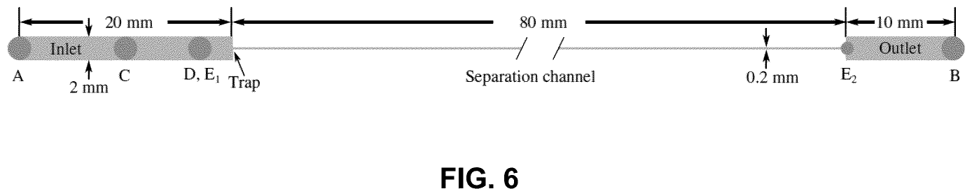

The microfluidic device shown in FIG. 1A of the present disclosure contains a number of features that enable an effective purification of a cell lysate. Besides the separation channel described earlier, these include inlet and outlet regions, and additional ports and electrodes. The outlet region serves an important function by separating the Luer connector from the channel. When fluid from the channel goes straight to the Luer (1), a large volume of protein and other contaminants remains trapped in the dead-zone around the Luer connector. When DNA is collected from the device, protein quickly follows, leading to much lower purity of the sample (A260/280<1.5). The additional electrodes (E1, E2) allow the field to be applied across the channel, so that only DNA is collected at the extraction port (D). The shape of the inlet region causes the flow to constantly flush both the injection port (C) and the extraction port (D), without any contamination at either port once the injection is finished. Keeping the extraction port near the trapped region makes the extraction quicker, but its precise location is not critical. FIG. 6 shows a similar design with a longer separation channel, which increases the yield.

Described herein are improved microfluidic traps and, in aspects, the successful extraction of DNA from E. coli. This iteration of the separation process has two key additions over the base operation which may be utilized. Electrodes placed in the channel itself 1) prevent contaminants from returning into the region containing purified DNA and 2) assist in collecting/recovering the purified DNA from the chip.

As illustrated in FIG. 1B, electrohydrodynamic migration drives DNA to accumulate at the walls of the separation channel, while all other components of the cell lysate remain uniformly distributed. In parabolic flows, the contaminants (ions, proteins, and cellular debris) are convectively eluted into the outlet region, while DNA near the walls moves electrophoretically in the opposite direction, towards the inlet region. Once DNA reaches the inlet region, the fields abruptly drop due to the expansion of the device. Over time this creates a region of concentrated DNA near the entrance to the separation channel, which is constantly being washed by the flowing buffer solution. The process is gentler than magnetic bead washing, where the DNA is held against the flow by the beads. Here, DNA undergoes a constant treadmilling process: trapped DNA (red region in FIG. 1B) diffuses from the walls of the device and is then picked up by the flow and convected into the separation channel. Without intending to be limiting, under weak shearing (less than 100 s⁻¹) the DNA migrates to the walls, whereupon it is returned to the inlet by electrophoresis. Same as with shear rates higher than 100 s⁻¹. The DNA is eluted as the field is reduced. The present disclosure demonstrated the feasibility of the device by recovering 10-20 ng of purified DNA in less than 30 minutes from 10 μL of E. coli lysate.

In certain aspects, devices, systems, and methods according to the present disclosure are particularly suitable for the isolation and/or extraction of nucleic acids from a biological sample having a size of 5 kb or greater than about 5 kb. In certain aspects, devices, systems, and methods according to the present disclosure are also suitable for the isolation and/or extraction of nucleic acids from a biological sample having a size of 5 kb or less than about 5 kb

### I. DEVICES

Described herein are microfluidic traps, and microfluidic traps comprising one or more electrodes.

In certain aspects, microfluidic traps according to the present disclosure can comprise: an inlet region having an injection port, extraction port, and a first electrode; an outlet region having an outlet port and a second electrode; and a microfluidic channel providing fluidic communication between the inlet region and outlet region.

In certain aspects, the first electrode and extraction port are in close proximity and separated from the injection port by an inlet distance.

In certain aspects, the second electrode and outlet port are separated by an outlet distance.

In certain aspects, the injection port, extraction port, and outlet port are in line with the longitudinal axis of the microfluidic channel.

In certain aspects, the first electrode and second electrode are constructed from or otherwise comprise a noble metal (for example, an electrode having a noble metal core). In certain aspects, the first electrode and second electrode comprise platinum. In other embodiments, electrodes may be constructed of or comprise other metal alloys like stainless steel, for example.

In certain aspects, the first electrode and second electrode are coated with a charge neutral polymer having a sufficiently small pore size to prevent passage of genomic DNA or other nucleic acids through the pores or from making contact with the electrode[s]. In embodiments, the electrodes are coated with agarose, polyacrylamide, agar, or other gel material used in protein and/or nucleic acid electrophoresis.

In certain aspects, the microfluidic channel is about 1 mm to about 100 cm long.

In certain aspects, the microfluidic channel has a square-shaped cross-section of about 0.5×0.5 microns to about 1×1 cm. In certain aspects, the microfluidic channel has a rectangular-shaped cross-section or a circular-shaped cross-section.

In certain aspects, the inlet distance is about 0.5 cm to about 5 cm.

In certain aspects, the outlet distance is about 0.5 cm to about 5 cm.

In certain aspects, the inlet region, outlet region, microfluidic channel, or any combination of any thereof are laser etched in acrylic. In certain aspects, the acrylic is bonded multilayer acrylic. In certain aspects, the inlet region, outlet region, microfluidic channel, or any combination of any thereof comprise one or more of glass, silicon, polydimethylsiloxane (PDMS), paper, a thermoplastic material, or a combination of any thereof. In certain aspects, the thermoplastic material is polycarbonate. In certain aspects, the inlet region, outlet region, microfluidic channel, or any combination of any thereof are bonded and layered. In certain aspects, the inlet region, outlet region, microfluidic channel, or any combination of any thereof are formed from a mold or machined.

In certain aspects, the inlet region further comprises an inlet flow adaptor.

In certain aspects, the outlet region further comprises an outlet flow adaptor.

In certain aspects, the inlet flow adaptor and outlet flow adaptor are each male or female luer connectors.

Additional details of microfluidic traps as described herein can be found, for example, in U.S. Patent Publication US20230381778A1, U.S. patent application Ser. No. 15/746,319, U.S. patent application Ser. No. 17/506,853, U.S. patent application Ser. No. 18/245,947, and International PCT Publication WO2017015468A1, the entire contents of all of which are incorporated by reference as if fully set forth herein.

### II. SYSTEMS

Described herein are systems. In certain aspects, systems comprise a microfluidic trap or device as described in section (I) above and exemplified in FIGS. 1A and 1C. Systems as described herein can further comprise a voltage generator, one or more fluid flow valves, and a fluid flow generator. In certain aspects, the voltage generator is any generator or amplifier capable of delivering a DC voltage of up to approximately 5 kV. In certain aspects, the one or more fluid flow valves are on-board valves, embedded microvalves, or 2-way miniature valves. In certain aspects, the one or more fluid flow valves are memetic miniature valves, MV-1 Embedded Microvalves, or ALine on-board diaphragm valves.

In certain aspects, systems can comprise a fluid source in fluidic communication with the inlet region of the microfluidic trap. In certain aspects, the fluid source comprises a buffer solution. In certain aspects, the buffer solution comprises Tris-borate-ethylenediaminetetraacetic acid (EDTA) (TBE), Tris-EDTA (TE), or Tris-acetate-EDTA (TAE). In certain aspects, the buffer solution comprises 0.25×TE. In certain aspects, the buffer solution comprises 0.25×TE diluted by a factor of 4.

In certain aspects, systems can further comprise a fluid collection device in fluidic communication with the outlet region of the microfluidic trap.

In certain aspects, systems can further comprise a flow valve in fluidic communication with the fluid source.

In certain aspects, the fluid flow generator is an active or passive fluid flow generator.

In certain aspects, the active fluid flow generator is a fluidic pump.

In certain aspects, the passive fluid flow generator is gravitational flow created by a difference in height between the fluid source and the fluid collection device relative to the microfluidic trap, wherein the fluid source is a greater height over the microfluidic trap than the fluid collection device.

### III. METHODS

Described herein are methods of trapping and/or detecting polyelectrolytes, charged polymers, and nucleic acids, for example, any deoxyribonucleic acid or any ribonucleic acid. Methods as described herein can comprise providing or administering a sample to a microfluidic trap and trapping nucleic acids of the sample. In certain aspects, devices, systems, and methods as described herein trap only genomic DNA or DNA molecules having a size greater than about 5 kb. In certain aspects, it is understood that charged molecules of other sizes can also be isolated, trapped, or otherwise detected, for example, DNA molecules with a size of about 5 kb, less than about 5 kb, or greater than about 5 kb.

In certain aspects, methods comprise establishing a fluid flow in a system as described herein; providing a first electric field across the longitudinal axis of the microfluidic channel with the voltage generator; injecting a biological sample comprising genomic DNA into the injection port of the system; waiting a first period of time; stopping the fluid flow; removing the first electric field; providing a second electric field across the first and second electrodes of the microfluidic trap with the voltage generator; waiting a second period of time; collecting the remaining nucleic acids from the extraction port of the microfluidic trap. In certain aspects, the fluid flow is unsteady. In certain aspects, the first electric field and/or the second electric field are unsteady.

In certain aspects, the biological sample comprises cell lysate comprising genomic DNA. In certain aspects, the first flow has a first flow rate up to about 500 microliters/minute, wherein the dimensions of the microfluidic channel determine the first flow rate.

In certain aspects, the first electric field has a strength of up to 500 V/cm.

In certain aspects, the polarity of the first electric field is such that the polarity attracts a polyelectrolyte or nucleotide to the inlet region.

In certain aspects, the first period of time is about 1 minute to about 5 hours, wherein the time depends on the length and cross section of the microfluidic channel.

In certain aspects, the second electric field has a strength of up to 500 V/cm.

In certain aspects, the polarity of the second electric field is such that the polarity attracts a polyelectrolyte or nucleotide to the inlet region.

In certain aspects, second period of time is about 1 minute to about 5 hours.

In certain aspects, the remaining nucleic acids have a size of greater than about 5 kb. In certain aspects, the remaining nucleic acids have a size less than or equal to about 5 kb. The skilled artisan would understand that the size cutoff may be varied according to various use cases.

### IV. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1: Microfluidic Purification of Genomic DNA

### i. Introduction

Microfluidic processing and analysis of DNA continues to be a vibrant area of research and development due to the reduced sample sizes and possibilities for integrating sample preparation and analysis into a single fully automated device (1-3). A cell lysate contains a number of contaminants that must be removed before any analysis of the DNA is possible. These include components native to the biological sample, such as polysaccharides in plant-based samples, or reagents used in the lysing process, such as salts and detergents (4, 5). In practice, purification is typically performed on relatively large samples (milliliter volumes) using liquid or solid-phase extraction methods, including magnetic beads that bind reversibly to the DNA (6). However, these methods are time-consuming and labor-intensive (7), involving multiple pipette transfers and centrifugation steps. The stresses created by centrifuging fragment individual DNA strands, which is undesirable when preparing samples for long-read sequencing (8, 9).

Microfluidic devices for DNA purification often miniaturize macroscopic techniques (7), such as gel electrophoresis (10) and solid-phase adsorption (11). Most microfluidic approaches to purification, including flow fractionation (12), electrokinetic trapping (13), and isotachophoresis (14), rely on the mobility contrast between nucleic acids and contaminants, such as proteins. However, achieving and maintaining the substantial concentration gradients necessary for effective separation, comparable to the purity levels obtained from commercial wash kits, is challenging. Microfluidic methods have yet to become widely adopted, due to inherent challenges such as dilute extracts and protein contamination. The increasing popularity of magnetic beads, despite their high cost and one-time use in many instances, reflects the difficulty of obtaining samples of high purity by alternative methods.

Here, a fundamentally different separation mechanism, based on the transverse migration of long polyelectrolyte molecules subjected to axial electric fields and flow, is utilized. It offers a more effective separation than existing methods relying on mobility contrasts between individual nucleic acids and contaminants.

Separation of DNA from the other components of the cell lysate occurs in the narrow channel (FIG. 1A) where the fields are highest. As illustrated in FIG. 1B, electrohydrodynamic migration drives DNA to accumulate at the walls of the separation channel, while all other components of the cell lysate remain uniformly distributed. In parabolic flows, the contaminants (ions, proteins, and cellular debris) are convectively eluted into the outlet region, while DNA near the walls moves electrophoretically in the opposite direction, towards the inlet region. Once DNA reaches the inlet region, the fields abruptly drop due to the 10-fold expansion of the device (FIG. 1A). Over time this creates a region of concentrated DNA near the entrance to the separation channel, which is constantly being washed by the flowing buffer solution. The process is gentler than magnetic bead washing, where the DNA is held against the flow by the beads. Here, DNA undergoes a constant treadmilling process: trapped DNA (red region in FIG. 1A) diffuses from the walls of the device and is then picked up by the flow and convected into the separation channel. Under weak shearing (less than 100 s-1) the DNA migrates to the walls, whereupon it is returned to the inlet by electrophoresis.

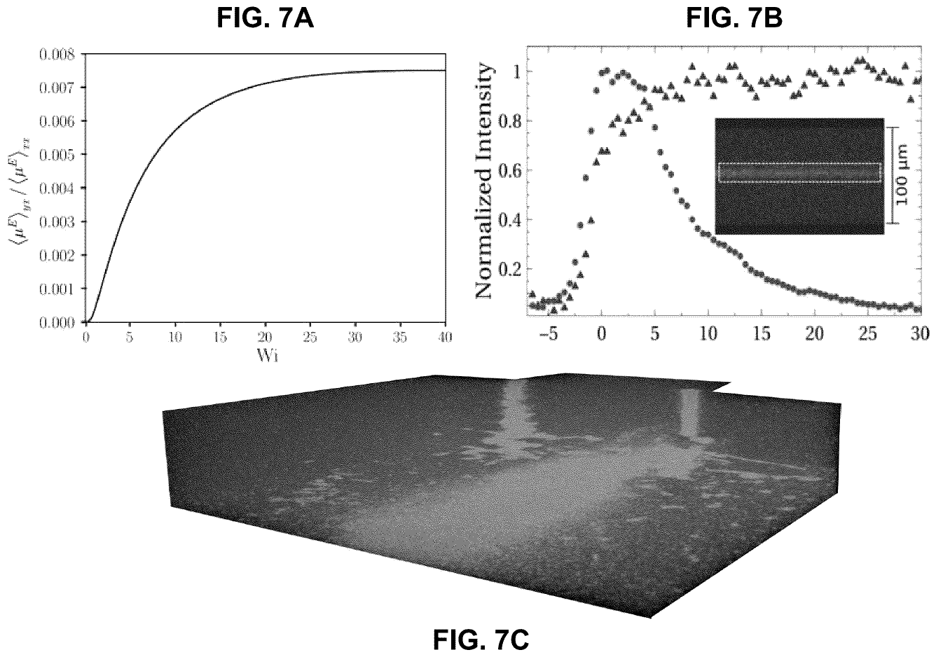

The mechanism for electrohydrodynamic migration was outlined in previous publications (15, 16). A polymer in a shear flow stretches into a cigar-like conformation (on average), which aligns at a small angle to the axis of the channel, as sketched in FIG. 1B. The slight rotation is crucial to the migration; if the stretched DNA was aligned exactly along the axis of the channel, it would simply follow the electric field. The loss of spherical symmetry makes the electrophoretic mobility anisotropic (17-20), and the axial electric field generates a component of the electrophoresis perpendicular to the field as well as parallel. In the configuration illustrated in FIG. 1B, the electrophoretic velocity of the DNA opposes the convective velocity from the flow, and produces a small transverse velocity of DNA towards the wall (17-20). Confocal images showing the distribution of DNA in a microfluidic device are shown in FIGS. 7A-7C. A theoretical analysis of polyelectrolyte migration is summarized in Example 2 (Polyelectrolyte migration).

Migration concentrates DNA in a narrow region near the walls (15), where it moves toward the inlet to the channel due to the dominance of the electrophoretic velocity over the (negligible) convective flow adjacent to the wall. Transverse migration distinguishes genomic DNA from other cell lysate components, which are eluted by the convective flow, regardless of their charge state. The key difference lies in the size and flexibility of DNA compared to other molecules in the lysate. For instance, proteins are small fairly rigid molecules, typically less 10 nm in size. In contrast, E. coli DNA is approximately 1.6 mm in length, with an equilibrium radius of about 10 μm. Rigid molecules tend to tumble in a shear flow, presenting a roughly spherical distribution on average, which limits their opportunity to migrate perpendicular to the field. Conversely, flexible polymers like DNA do not so much tumble as repeatedly collapse back to a near spherical configuration and then quickly elongate again into the cigar shape with the same shear-dependent orientation. This persistent reorientation and stretching creates a consistent driving force for transverse electrophoresis.

In situ fluorescence measurements (16) have suggested that mixtures of A-DNA with physiological concentrations of BSA (bovine serum albumin) can be separated by electrohydrodynamic migration. Here reconfigured microfluidic devices, which allow samples of purified DNA to be collected and analyzed by standard analytical techniques: fluorometry, gel electrophoresis, and PCR (polymerase chain reaction) amplification, were tested. To this end, microfluidic devices were constructed using laser cutting and thermal bonding of acrylic sheets (16). These devices were used to extract purified DNA from lysates of E. coli and human cells, and from mixtures of A-DNA and BSA (bovine serum albumin). It was demonstrated that electrohydrodynamic separation (EHS) produces DNA samples of sufficient purity for PCR amplification, indicating its potential for various genetic analysis techniques, including longread sequencing. Gel electrophoresis reveals that the purified DNA shows minimal fragmentation up to 60 kbp. These characteristics would be beneficial in long-read sequencing applications.

### ii. Results

Microfluidic purification of genomic DNA (E. coli K12) from a cell lysate is reported. The experiment was set up as illustrated in FIG. 1C, with a 0.25×TE buffer solution flowing into the device through port A and out through port B (FIG. 1A). E. coli cells were lysed with a solution of lysozyme and proteinase K in SDS (sodium dodecyl sulfate) solvent, before being injected into the separation channel through port C (see FIG. 1A). The injected solution is subjected to a constant flow and electric field for about 15 minutes, during which time the proteins, cellular debris, and salts are flushed out, leaving purified DNA suspended in the 0.25×TE buffer. Then the electric field is turned off and the flow is stopped by closing the outlet valve to the reservoir (FIG. 1C). A positive potential is applied between the electrodes E1 and E2 to draw the DNA to the extraction port, which takes an additional 10 minutes. A 10 μL sample is drawn from the extraction port, either with a syringe or a pipette.

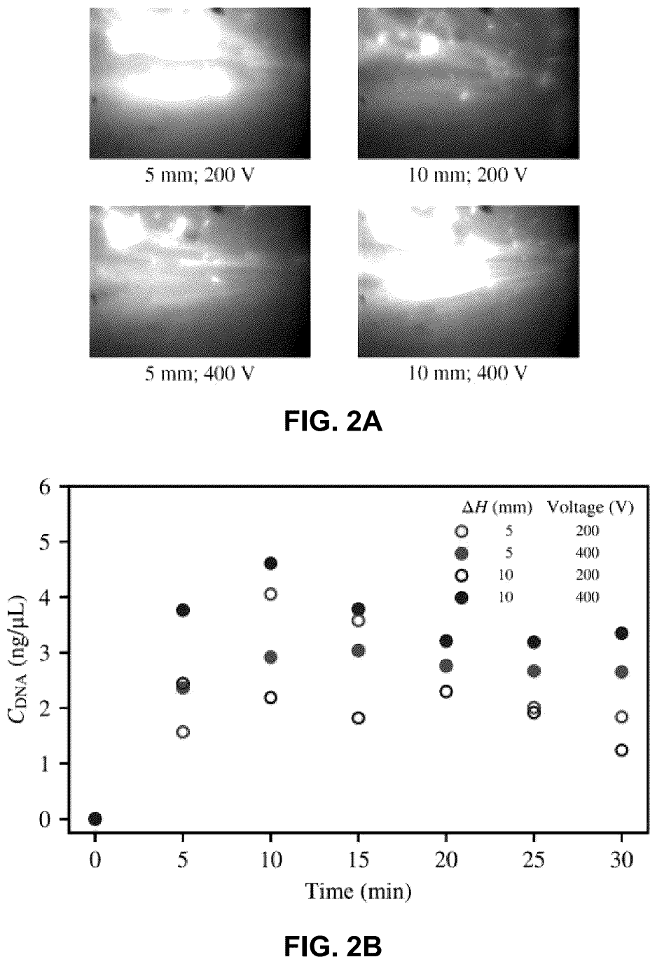

E. coli extraction. Fluorescence imaging was used to measure the in situ DNA concentration in the region of the trap, as illustrated in FIG. 2A. Images from regions of highest concentration (near the entrance to the separation channel) are shown for different flow rates and field strengths. The amount of DNA retained near the separation channel inlet depends on the shear rate and electric field in the separation channel (FIG. 2A). The maximum trapping occurs at the highest field strengths (ΔH=10 mm, V=400 V), but it is also important that the field and flow are correlated (15). The image at low flow and low voltage (ΔH=5 mm, V=200 V) shows more trapping than either of the mixed cases (ΔH=5 mm, V=400 V or ΔH=10 mm, V=200 V). The average intensity in the viewing window (over background) was converted to concentration using standard solutions (see Example 2: Methods). FIG. 2B shows the average concentration of E. coli DNA near the inlet to the separation channel (FIG. 1A) as a function of time. Results for individual samples show that DNA remains trapped in the device for at least half an hour, with concentrations in the range 2-5 ng/μL. During this time, the device (total volume of approximately 30 μL) is flushed about 5 times with fresh TE buffer solution.

TABLE 1
Concentrations and Cq values for E. coli DNA after
microfluidic purification of a cellular lysate.

| Item | Qubit (ng/μL) | C_{q} |
|---|---|---|
| E. coli (l) 1 | 1.28 | 15 |
| E. coli (l) 2 | 1.67 | 15 |
| E. coli (l) 3 | 1.31 | 16 |
| E. coli (u) 1 | 1.65 | 14 |
| E. coli (u) 2 | 1.74 | 15 |
| E. coli (μL) 1 | 2.94 | 12 |
| E. coli (μL) 2 | 3.58 | |
| E. coli (μL) 3 | 4.06 | |
| E. coli (μL) 4 | 2.26 | |
| E. coli lysate | 49.3 | ∞ |

Both labeled (l) and unlabeled (u) samples were purified and the concentration was measured by a Qubit. Additional (unlabeled) samples were extracted using a longer (80 mm) separation channel (uL). Note that Qubit measurements of labeled samples were corrected for excess fluorescence from added YOYO-1 (see Example 2: Methods).

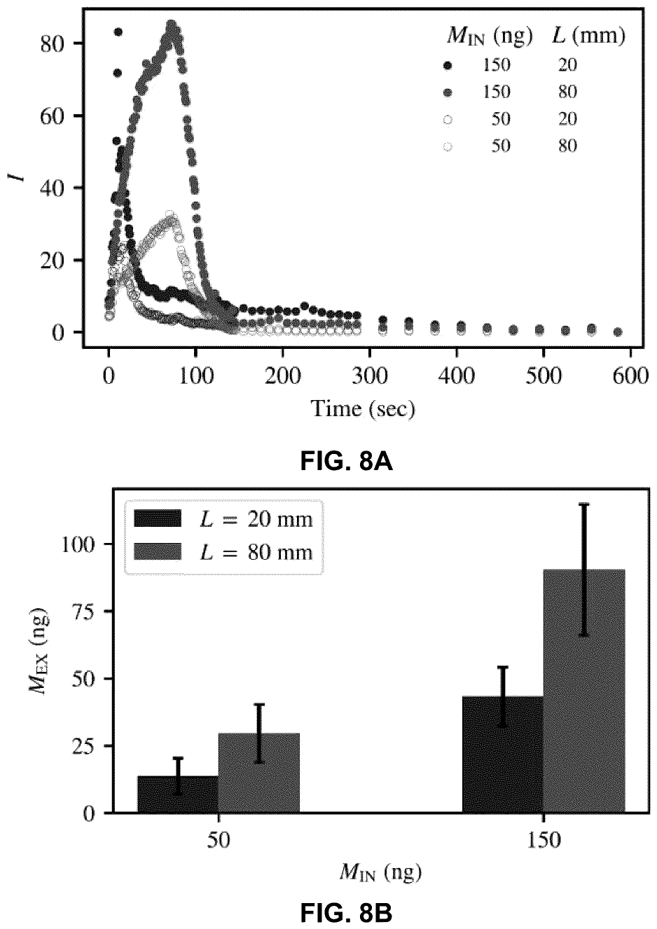

Concentrations of 10 μL samples of purified E. Coli DNA are reported in Table 1. The first five extracts were obtained with the 22 mm separation channel (FIGS. 1A-1C), but a series of in situ measurements of DNA trapping (see Example 2: Mass balance) showed that only about 20% of the injected DNA was being trapped by the device (FIGS. 8A-8B). Visual observations confirmed that significant concentrations of DNA were present along the length of the separation channel, suggesting that trapping could be improved by increasing the length of the separation channel. Additional extractions (4) were made with a new device with an 80 mm separation channel (FIG. 6). Larger pressure drops and voltages (ΔH=25 mm, V=600 V) were used, which create approximately the same flow rate and electric field in the separation channel as before. There is some variation in the amounts extracted due to uncertainties in the positioning of the syringe, but both devices extract significant amounts of DNA; up to 17 ng in the shorter channel and 40 ng in the longer one. The new device was used to extract higher concentration samples for the gel electrophoresis, but the other measurements are not concentration sensitive.

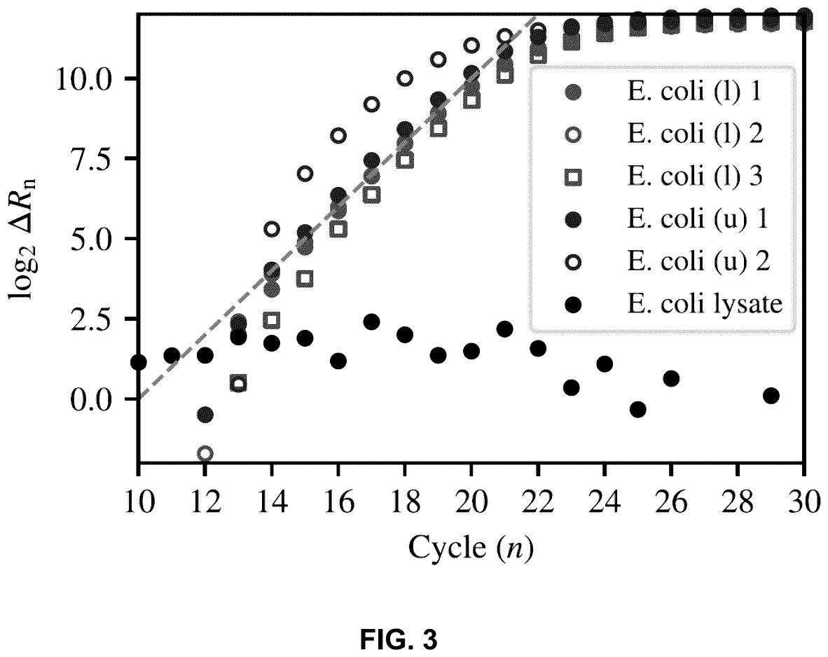

PCR amplification. PCR amplification is a key step for most DNA analysis, but cations, proteins, and other components of the lysate can inhibit the process. Small quantities of SDS (>0.005%) have been shown to completely inhibit PCR (21, 22), while EDTA is also an effective PCR inhibitor at concentrations in excess of 0.5 mM (5, 23). The lysis buffer solution contained 0.17% SDS and 12.8 mM EDTA, which explains why the lysate itself shows no PCR amplification (black symbols in FIG. 3). However, the samples extracted from the microfluidic device (colored symbols) all amplify rapidly with Cq=15.

FIG. 3 shows the PCR signal (minus the background) as a function of the cycle number for the five samples extracted with the 22 mm channel (blue and red symbols) and for the original cell lysate (black circles). A logarithmic scale was used to emphasize the exponential growth of concentration. All five of the extracted samples (Table 1) amplified rapidly, as illustrated in FIG. 3, in all cases reaching the default threshold in 16 cycles. The unlabeled samples (blue) amplified faster than the labeled ones (red), consistent with YOYO-1 being a mild PCR inhibitor (24). The Cq values for the extracted samples (Table 1) compare favorably with those from commercial wash kits, which are typically around Cq=30 (25).

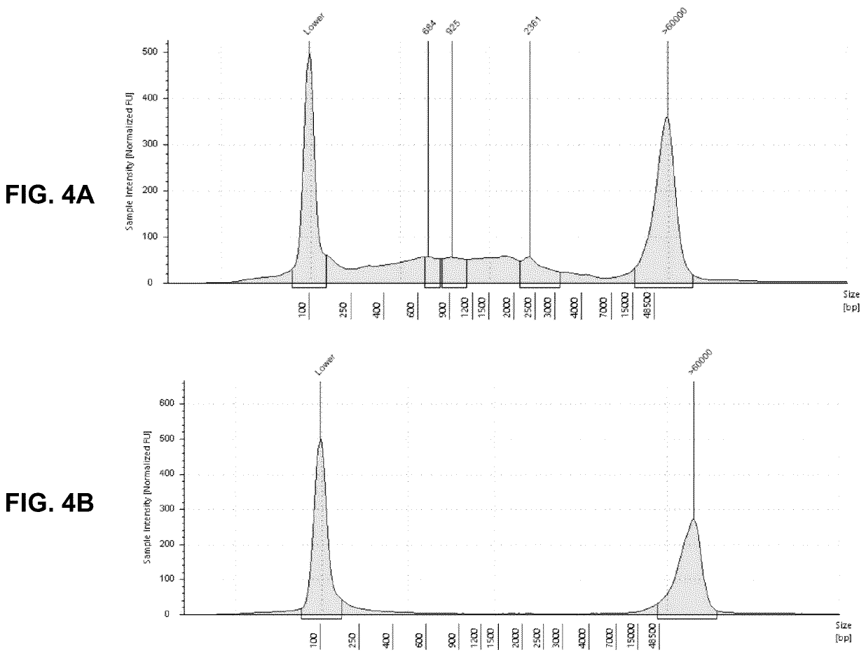

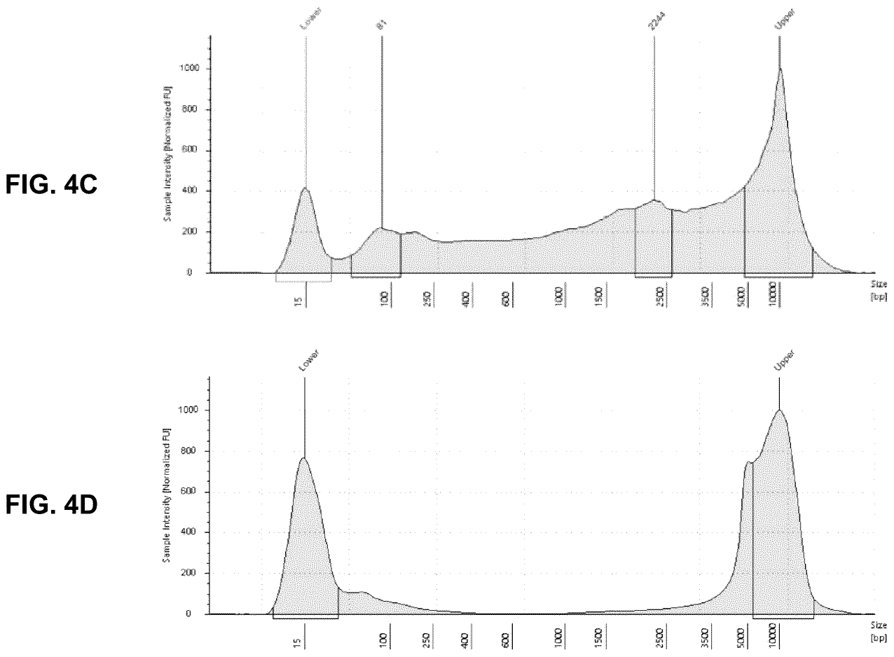

Length distribution. DNA fragmentation during lysing and purification undermines the potential of long-read sequencing technologies to provide more comprehensive genomic data. The microfluidic extraction described here uses a very low shear rate, less than 100 s-1, which suggests samples with less additional fragmentation can be obtained as compared to conventional protocols, which require centrifuging the sample. To confirm this, gel electrophoresis was run on four different samples (FIGS. 4A-4D), two of them using the Genomic ScreenTape (fragment lengths up to 60 kbp, sensitivity 0.5 ng/μL) and two of them using the High Sensitivity DNA ScreenTape (fragment lengths up to 5 kbp, sensitivity 5 pg/μL).

The cell lysate (FIG. 4A) shows significant fragmentation, with a broad distribution of lengths. However, the purified sample (FIG. 4B) shows just a single peak near the maximum readout length of the TapeStation (60 kbp), suggesting that the extraction process does not produce significant fragmentation up to 60 kbp. Nevertheless, because of the difference in concentrations, the data in FIGS. 4A & FIG. 4B do not show unambiguously that fragmentation is reduced by the purification process. To clarify the extent to which fragmentation of the original lysate can be reduced by purification, we diluted the lysate to match the concentration of the purified DNA. Electropherograms for the original lysate and the purified sample at similar concentrations are shown in FIG. 4C and FIG. 4D. These measurements used the High Sensitivity ScreenTape, which limits the range of lengths to 5 kbp but greatly enhances sensitivity. It is clear that fragmentation has been significantly reduced, particularly below 3.5 kbp.

Protein removal. Many microfluidic extractions are based on the differential mobility of proteins and DNA (10, 12-14), but their ability to reduce protein concentrations is limited. A 100 fold reduction in protein concentration is typical (12), resulting in high protein concentrations (10) and poor A260/A280 ratios. The extraction proposed here is convective in nature, with much higher separation potential.

A reasonably pure sample of E. coli DNA (e.g. A260/280>1.7) would require protein levels below 5 ng/μL, given the concentration of the DNA extract (≈2 ng/μL). However, this is below the detection limit of Qubit fluorometry (12.5 ng/μL). Therefore, to demonstrate the potential for electrohydrodynamic separation of DNA and proteins, 10 μL mixtures of A-DNA (15 ng/μL) and bovine serum albumin (30 μg/μL) were injected, using the BSA as a proxy for proteins. The BSA was spiked with 5% FITC (fluorescein isothiocyanate conjugate) labeled protein to provide a strong fluorescence signal, allowing concentrations as small as 0.1 ng/μL to be detected; this makes the measurement about 100 times more sensitive than the Qubit.

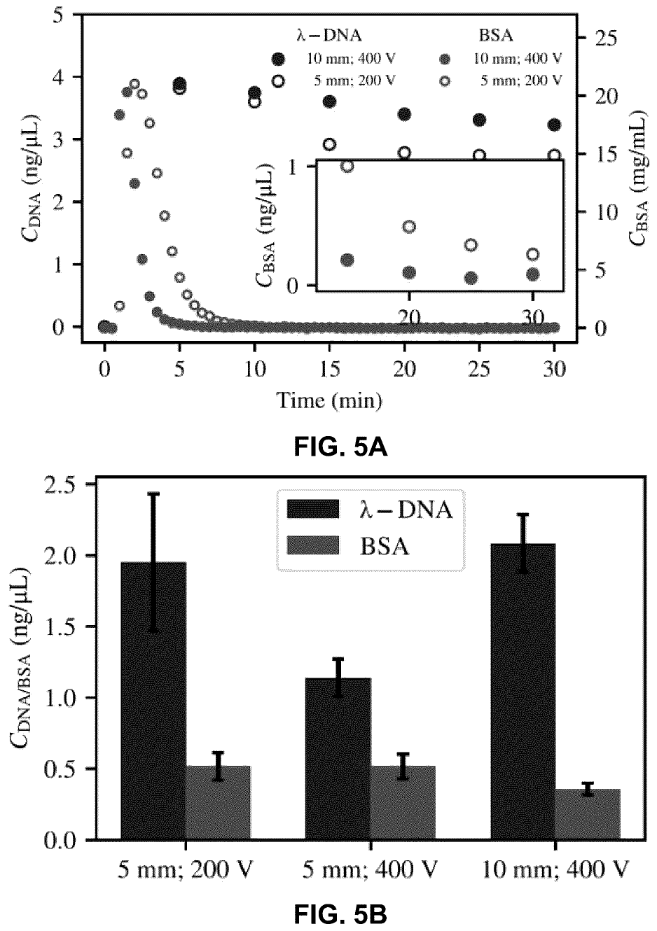

FIG. 5A shows the concentrations of DNA (blue symbols) and BSA (red symbols) in the region of the trap (FIG. 1A). The DNA concentration rises rapidly after sample injection, reaching a maximum of 4 ng/μL after less than 5 minutes. Over the next 15 minutes the concentration in the trap slowly declines to a steady-state value of 3 ng/μL for the conditions used in these experiments. Based on calibrations (Example 2: Methods), the mean fluid velocity was estimated at 3.8 mm/s and the electrophoretic velocity at 0.11 mm/s. Maximal trapping occurs when the mean flow is about 35 times the electrophoretic velocity.

The BSA concentration also peaks rapidly, but starts to decline steeply after 2-3 minutes because the trapping mechanism does not apply to small rigid molecules. The concentration of protein in the injected sample (30 μg/μL) is much larger than the DNA concentration (15 ng/μL), but in the trap it diminishes rapidly as the BSA is flushed out, dropping below 1 ng/L after 15 minutes and finishing below 0.25 ng/μL.

The DNA concentration in the extract (FIG. 5B) is about 2 ng/μL indicating that a significant fraction of the trapped DNA can be recovered, even by a manual extraction with a syringe. The yield (20 ng) is similar to the extraction of E. coli in a channel of similar length. The BSA concentration in the extract is reduced by 5 orders of magnitude, corresponding to an A260/280 ratio of 1.9 (see Example 2: Methods). This level of sample purity is consistent with the rapid PCR amplification of the E. coli lysate.

### iii. Discussion

The mechanism underlying electrohydrodynamic separation bears a resemblance to field flow fractionation (FFF) (26, 27). In both cases, a transverse force, balanced by diffusion, sets up a concentration profile across the channel. The parabolic velocity profile then drives a differential elution of the different species. However, in FFF the separation of the species is limited by diffusional spreading of the concentration bands of the different species, whereas in electrohydrodynamic separation (EHS) only the target species (DNA) is concentrated near the wall. This creates a complete separation of the DNA, which returns to the inlet by electrophoresis, and the remaining components, which are convected to the outlet. It is worth noticing that although DNA has a transverse component of its velocity, the fields are strictly axial. The transverse motion is caused by the asymmetry in the electrophoretic mobility of the DNA which is stretched and rotated by the shear flow.

A microfluidic FFF was shown to reduce the protein concentration by two orders of magnitude (12), whereas the results in FIGS. 5A-5B show a five order of magnitude reduction by EHS. Gel extraction, whether microfluidic or macroscopic, leads to large protein to DNA ratios, approximately 15 times as much protein as DNA (10). By contrast, after EHS the DNA concentration is about 7 times the protein concentration. Another related separation mechanism (also called electrohydrodynamic) makes use of the normal stresses generated by the shear of a viscoelastic fluid (28). Here the electric field serves to move the DNA electrophoretically with respect to the fluid, causing migration towards the wall when the electrophoresis opposes the flow. Different particle shapes and sizes will migrate to different extents allowing for some degree of separation, but the mechanism is not specific to a long polyelectrolyte (as EHS is), even spherical particles migrate in a viscoelastic fluid.

The rapid PCR amplification (FIG. 3) shows that EHS is effective in removing PCR inhibitors, such as SDS, enzymes, and proteins. The lysis buffer contained 0.17% SDS, which severely inhibits PCR and is likely the reason why untreated lysate cannot be amplified (black circles in FIG. 3). However, after EHS the DNA rapidly amplifies, measurably doubling every cycle after 13-14 cycles. The absence of gels, beads, posts, membranes, or other kinds of confinement, limits the shear during extraction to a maximum of 100 s-1. This is well below the critical shear for fragmentation, which is about 3000 s-1 (29). Comparing High Sensitivity electropherograms of extracted DNA with the original lysate (FIGS. 4C-4D) shows that fragments below 3 kbp are eliminated and the concentration of fragments up to 5 kbp is significantly reduced. EHS simplifies sample preparation for longread sequencing by combining DNA purification and long DNA isolation into a single step. This approach mitigates the need for additional gel electrophoresis-based size selection systems (30, 31).

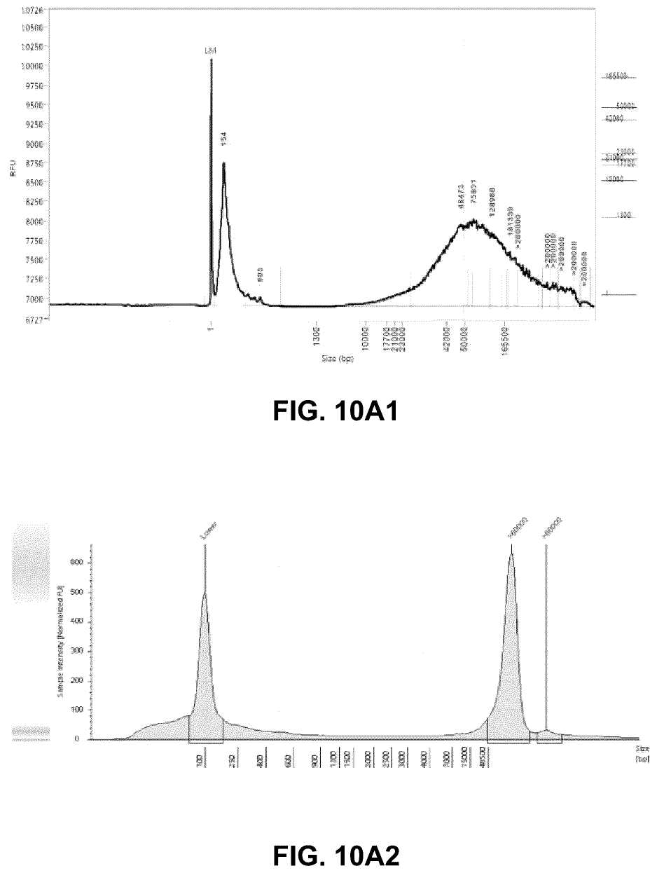

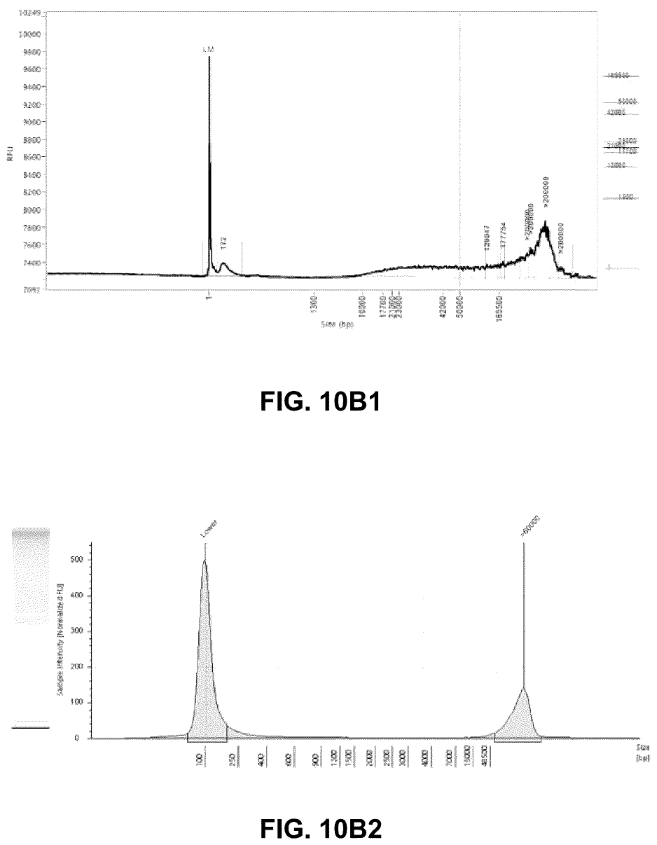

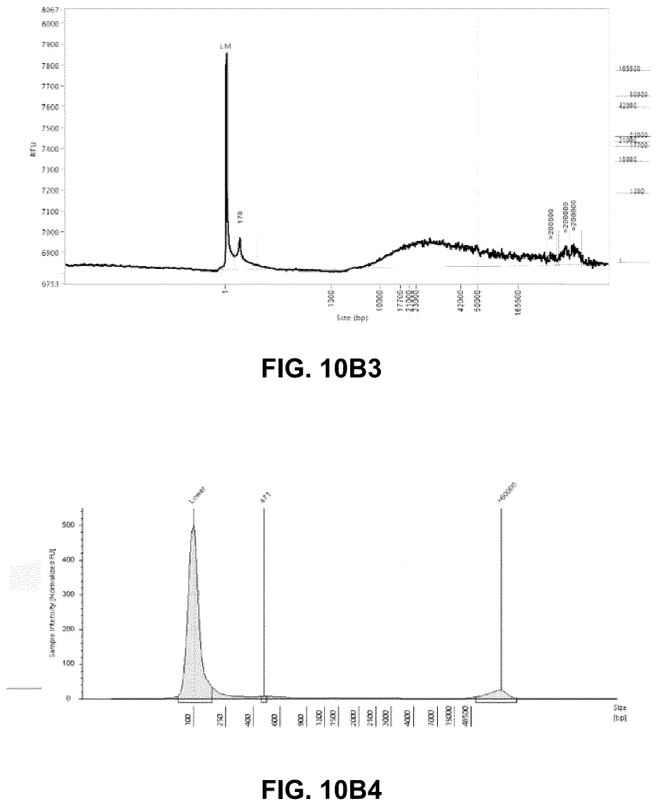

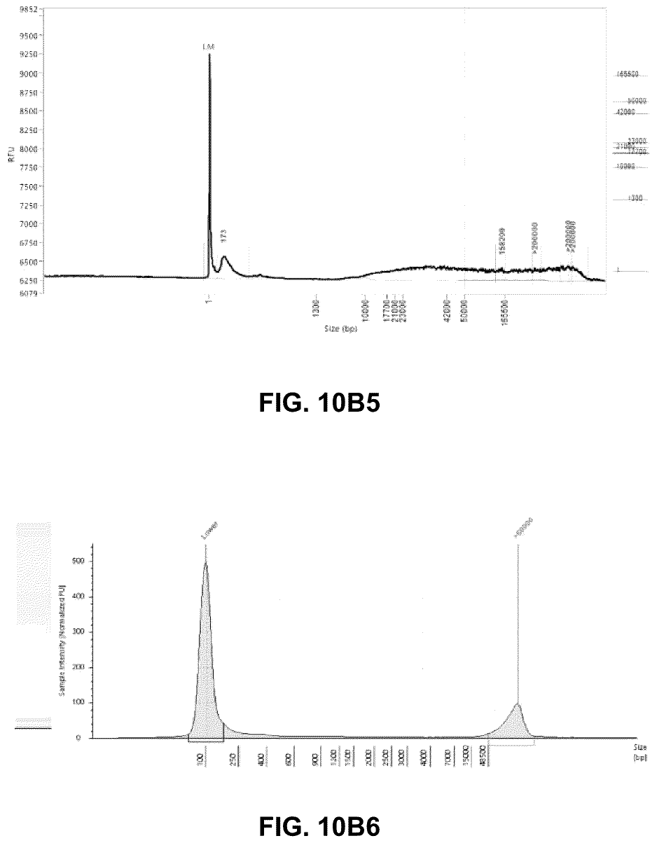

Electrohydrodynamic separation is applicable to mammalian DNA as well. FIGS. 10A1-10B6 show trapping of human DNA in the 80 mm channel. Several extracts were obtained, with concentrations up to 5 ng/μL. Femto Pulse (FIGS. 10A1-10B6) shows a reduction in concentration of shorter fragments (less than 50 kbp) in comparison with the lysate, while longer fragments are preserved.

### iv. Conclusions

EHS can extract DNA with comparable purity to magnetic bead extraction kits and possibly with less fragmentation because the polymer is not held in tension by the flow. The high level of sample purity (calculated A260/A280 ratios of 1.9) is due to the repeated washing of the trapped DNA and the absence of any additives to the 0.25×TE buffer. The trapping time (15 minutes) flushes about 150 μL of buffer solution, enough to wash the sample volume (≈10 μL) 15 times. At a cost of about $1 per copy the device is easily replaced when it is essential to avoid cross contamination. On the other hand, replacing the magnetic beads for every extraction incurs a significant cost. Although the maximum yield from the device (40 ng in the 80 mm channel) exceeds most microfluidic separations (typically picograms to nanograms), it does not yet match the yields of bead extraction kits (in the range of μg).

### v. Materials and Methods

Microfluidic devices were constructed using laser-cut acrylic sheets that were thermally bonded to form fluid channels. Ports for Luer connectors and electrodes were added as illustrated in FIG. 1A and sealed with silicone. The device was mounted on an epifluorescence microscope and attached with Luer connectors to the inlet and outlet reservoirs. Fluid flow was generated by adjusting the height of one of the reservoirs and a voltage difference was applied to the fluid in the reservoirs.

Strands of DNA (λ) were obtained from a commercial preparation, and longer strands were prepared by lysing E. Coli (K12) and human HEK 293 cells. Device performance was quantified by in situ fluorescence measurements and external characterizations, including PCR and gel electrophoresis. Further information on the devices, testing protocols, sample generation, and quantification methods are provided in Example 2 below.

### vi. References for the Present Example

1. F. Cui, M. Rhee, A. Singh, A. Tripathi, Microfluidic sample preparation for medical diagnostics. Annu. Rev. Biomed. Eng. 17, 267-286 (2015).
2. D. Obino et al., An overview on microfluidic systems for nucleic acids extraction from human raw samples. Sensors 21, 3058 (2021).
3. K. Yamamoto, N. Ota, Y. Tanaka, Nanofluidic devices and applications for biological analyses. Anal. Chem. 93, 332-349 (2021).
4. L. Rossen, P. Nørskov, K. Holmstrøm, O. F. Rasmussen, Inhibition of PCR by components of food samples, microbial diagnostic assays and DNA-extraction solutions. Int. J. Food Microbiol. 17, 37-45 (1992).
5. C. Schrader, A. Schielke, L. Ellerbroek, R. Johne, PCR inhibitors-Occurrence, properties and removal. J. Appl. Microbiol. 113, 1014-1026 (2012).
6. X. Ye, B. Lei, The current status and trends of DNA extraction. Bioessays 45, 2200242 (2023).
7. L. Xia, G. Li, Recent progress of microfluidic sample preparation techniques. J. Sep. Sci. 46, 2300327 (2023).
8. G. A. Logsdon, M. R. Vollger, E. E. Eichler, Long-read human genome sequencing and its applications. Nat. Rev. Genet. 21, 597-614 (2020).
9. K. Shafin et al., Nanopore sequencing and the Shasta toolkit enable efficient de novo assembly of eleven human genomes. Nat. Biotechnol. 38, 1044-1053 (2020).
10. P. Agrawal, K. D. Dorfman, Microfluidic long DNA sample preparation from cells. Lab Chip 19, 281-290 (2019).
11. T. Lund-Olesen, M. Dufva, M. F. Hansen, Capture of DNA in microfluidic channel using magnetic beads: Increasing capture efficiency with integrated microfluidic mixer. J. Magn. Magn. Mater. 311, 396-400 (2007).
12. C. Zhang, G. Sun, S. Senapati, H. C. Chang, A bifurcated continuous field-flow fractionation (BCFFF) chip for high-yield and high-throughput nucleic acid extraction and purification. Lab Chip 19, 3853-3861 (2019).
13. W. Ouyang, Z. Li, J. Han, Pressure-modulated selective electrokinetic trapping for direct enrichment, purification, and detection of nucleic acids in human serum. Anal. Chem. 90, 11366-11375 (2018).
14. A. Persat, L. A. Marshall, J. G. Santiago, Purification of nucleic acids from whole blood using isotachophoresis. Anal. Chem. 81, 9507-9511 (2009).
15. M. Arca, A. J. C. Ladd, J. E. Butler, Electro-hydrodynamic concentration of genomic length DNA. Soft Matter 12, 6975-6984 (2016).
16. B. E. Valley, A. D. Crowell, J. E. Butler, A. J. C. Ladd, Electro-hydrodynamic extraction of DNA from mixtures of DNA and bovine serum albumin. Analyst 145, 5532-5538 (2020).
17. J. E. Butler, O. B. Usta, R. Kekre, A. J. C. Ladd, Kinetic theory of a confined polymer driven by an external force and pressure-driven flow. Phys. Fluids 19, 113101 (2007).
18. H. Pandey, P. T. Underhill, Coarse-grained model of conformation-dependent electrophoretic mobility and its influence on DNA dynamics. Phys. Rev. E 92, 052301 (2015).
19. A. J. C. Ladd, Electrophoresis of sheared polyelectrolytes. Mol. Phys. 1, 3121-3133 (2018). 20. D. I. Kopelevich, S. He, R. J. Montes, J. E. Butler, Mesoscopic models for electrohydrodynamic interactions of polyelectrolytes. J. Fluid Mech. 915, A59 (2021).
21. T. Demeke, G. R. Jenkins, Influence of DNA extraction methods, PCR inhibitors and quantification methods on real-time PCR assay of biotechnology-derived traits. Anal. Bioanal. Chem. 396, 1977-1990 (2010).
22. Y. Nakanishi et al., Letter to the editor: Speedy plant genotyping by SDS-tolerant cyclodextrin-PCR. Plant Cell Physiol. 63, 1025-1028 (2022).
23. W. A. Al-Soud, P. Radstrom, Purification and characterization of PCR-inhibitory components in blood cells. J. Clin. Microbiol. 39, 485-493 (2001).
24. H. Gudnason, M. Dufva, D. Bang, A. Wolff, Comparison of multiple DNA dyes for real-time PCR: Effects of dye concentration and sequence composition on DNA amplification and melting temperature. Nucleic Acids Res. 35, e127 (2007).
25. A. Psifidi et al., Comparison of eleven methods for genomic DNA extraction suitable for large-scale whole-genome genotyping and long-term DNA banking using blood samples. PLoS ONE 10, e0115960 (2015).
26. F. A. Messaud et al., An overview on field-flow fractionation techniques and their applications in the separation and characterization of polymers. Prog. Polym. Sci. 34, 351-368 (2009).
27. J. Janca, Field-Flow Fractionation (CRC Press, 2022).
28. H. Ranchon et al., DNA separation and enrichment using electro-hydrodynamic bidirectional flows in viscoelastic liquids. Lab Chip 16, 1243-1253 (2016).
29. Y. Qiao, Z. Ma, C. Onyango, X. Cheng, K. D. Dorfman, DNA fragmentation in a steady shear flow. Biomicrofluidics 16, 054109 (2022).
30. A. Jones et al., High-molecular weight DNA extraction, clean-up and size selection for long-read sequencing. PLoS ONE 16, e0253830 (2021).
31. R. Kalendar, K. I. Ivanov, O. Samuilova, U. Kairov, A. A. Zamyatnin Jr, Isolation of high-molecularweight DNA for long-read sequencing using a high-salt gel electroelution trap. Anal. Chem. 95, 17818-17825 (2023).

### Example 2: Additional Details of Example 1

### i. Materials and Methods

Device design. The microfluidic device shown in FIG. 1A of the present disclosure contains a number of features that enable an effective purification of a cell lysate. Besides the separation channel described earlier, these include inlet and outlet regions, and additional ports and electrodes. The outlet region serves an important function by separating the Luer connector from the channel. When fluid from the channel goes straight to the Luer (1), a large volume of protein and other contaminants remains trapped in the dead-zone around the Luer connector. When DNA is collected from the device, protein quickly follows, leading to much lower purity of the sample (A260/280<1.5). The additional electrodes (E1, E2) allow a field to be applied across the channel, so that only DNA is collected at the extraction port (D). The shape of the inlet region causes the flow to constantly flush both the injection port (C) and the extraction port (D), without any contamination at either port once the injection is finished. Keeping the extraction port near the trapped region makes the extraction quicker, but its precise location is not critical. FIG. 6 shows a similar design with a longer separation channel, which increases the yield.

Device fabrication. The microfluidic design was modeled using Rhino 5 software and manufactured from three PMMA (polymethyl methacrylate) sheets with a laser cutter (Trotec—Speedy 360). Ports for accessing the microfluidic device were cut through the top sheet (thickness 1.6 mm), the designs shown in FIG. 1A and FIG. 6 was cut through the middle sheet (0.2 mm thickness), and a solid sheet (thickness 0.2 mm) was used to seal the bottom of the channel. The sheets were thermally bonded with a heat press. After confirming bonding, luer connectors were epoxied to the top sheet above the inlet and outlet ports (A and B in FIG. 1A and FIG. 6). The injection, extraction, and electrode ports (C, D, E₁, and E₂ were sealed with silicone, and platinum wire electrodes were inserted into ports E1 and E2 after the silicone set.

Cell culture and lysis. E. coli K12 strain was cultured in a sterilized liquid lysogeny broth medium for 16 hours at 37° C. After culturing, cells were separated from the liquid culture by centrifugation. The cell pellet was weighed and resuspended in a suspension buffer containing 50 mM Tris-HCl, 50 mM NaCl, and 5% glycerol, using 7 mL of buffer per gram of wet cell weight. rLysozyme (Novagen) was added at 4 μg/ml of suspension buffer. The mixture was incubated at room temperature for 20 minutes, after which an equal volume of SDS lysis buffer (0.3% SDS, 100 mM Tris-HCl, 20 mM EDTA, 0.3 M NaCl) was added along with proteinase K (Thermo Fisher Scientific) to a final concentration of 1 mg/mL. The suspension was incubated at 55° C. for 30 minutes, followed by cooling to room temperature. DNA concentration was measured using a Qubit fluorometer. For certain experiments, YOYO-1 green fluorescent stain (491/509 nm excitation/emission) was added to enable DNA visualization during purification at a ratio of one dye molecule per four DNA base pairs. To prevent SDS from inhibiting YOYO-1 binding to DNA, a lower SDS concentration (0.15%) was used, following established protocols (2).

Frozen HEK 293 (human embryonic kidney 293) cells were thawed at room temperature and separated by centrifugation. RIPA (Research Products International) lysis buffer was added at a ratio of 1 mL per 40 mg of cell pellet. SDS lysis buffer (2% SDS, 100 mM Tris-HCl, 20 mM EDTA, 0.3 M NaCl) was added at 70 μL for each 1 mL of RIPA buffer, and proteinase K was added to a final concentration of 1 mg/mL.

DNA trapping and extraction. An 0.25×TE (Tris-EDTA) buffer solution runs through the device for the duration of the experiment, driven by a height difference between the two reservoirs (FIG. 1C). An electric potential difference is introduced between the inlet and outlet reservoirs creating the conditions for DNA trapping. Different heights and voltages were used to control the flow rate and electric field in the separation channel. The average fluid velocity and the electrophoretic velocity of the DNA were calibrated against the height and voltage differences using particle image velocimetry within the separation channel.

For each experiment, 10 μL of lysate was injected into the device with the background buffer solution running. After purifying the DNA for 15 minutes, the convective flow was halted by closing the outlet valve and the potential difference between the reservoirs was set to zero. The trapped DNA was moved to the extraction port by applying a potential difference of 80 V between electrodes E1 and E2 (FIG. 1A) for a period of 10 minutes. Samples labeled with YOYO-1 were used to develop an extraction protocol, but unlabeled samples were used in subsequent experiments.

Qubit measurements. DNA concentrations in the E. coli lysate were measured by a Qubit 4 Fluorometer (Thermo Fisher Scientific). Purified samples (1 μL) were mixed with 199 μL of working solution from the Qubit dsDNA Broad-Range Assay Kit (Thermo Fisher Scientific). Samples were collected for both unlabeled and labeled E. coli DNA. Measurements of YOYO-1 labeled DNA had to be corrected due to the additional fluorescence of the label. Calibrations indicated that Qubit measurements on labeled samples should be divided by 2.5; this gives the value reported in Table 1.

Fluorescence calibration of DNA and BSA. In situ concentrations (FIGS. 2A-2B, and FIG. 5A) were measured from the mean fluorescence intensity in the viewing window. DNA was labeled with intercalating YOYO dyes at a ratio of one dye molecule per four base pairs. E. coli DNA was labeled with green-emitting YOYO-1 (491/509 nm), while A-DNA used orange-emitting YOYO-3 (612/631 nm) to contrast with green-emitting FITC-labeled BSA. BSA was marked with FITC (fluorescein isothiocyanate) at 5% w/w, (494/520 nm). The fluorescence from YOYO-3-labeled DNA and FITC-labeled BSA can then be independently measured using green and blue excitation filters. The intensity measurements over the background signal were converted to concentrations using standard solutions of (labeled) λ-DNA and FITC BSA.

The concentration of samples extracted from the microfluidic device (FIG. 5B) were characterized using a fluorescence well. The well has a volume of 10 μL and was calibrated using standard solutions of A-DNA labeled with YOYO-3 and BSA labeled with FITC.

A260/A280 ratios. The extracted samples were too dilute for UV spectral analysis, but we can predict the A260/A280 ratios of the extracts from the concentrations of DNA and BSA (FIG. 5B). A simple formula for the A260/A280 ratio of the extract is:

$$R = \frac{{Ac}_{DNA} + {c}_{BSA} {R}_{BSA}}{{Ac}_{DNA} / {R}_{DNA} + {C}_{BSA}} \qquad [ eq . 1 ]$$

where R_{DNA}=1.9 and R_{BSA}=0.57 are the A260/A280 ratios of pure samples of DNA and BSA, and A=30 is the ratio of the extinction coefficients of DNA (at 260 nm) and BSA (at 280 nm). The extracts in FIG. 5B have A260/A280 ratios in the range 1.87-1.89.

qPCR. PCR was conducted using a C1000 Touch Thermal Cycler (Bio-Rad) with an initial denaturation step at 98_C for 3 minutes, followed by cycling at 98° C. for 5 seconds and 60° C. for 10 seconds. To amplify E. coli DNA, the forward primer (F): GCTACAATGGCGCATACAAA (SEQ ID NO:1) and Reverse primer (R): TTCATGGAGTCGAGTTGCAG (SEQ ID NO:2) (3) obtained from Integrated DNA Technologies (IDT) were used. Each reaction mixture contained 10 μL of SsoAdvanced Universal SYBR Green Supermix (Bio-Rad), 1 μL each of forward and reverse primer, and 8 μL of template DNA sample.

Electrophoresis. DNA length analysis was performed using the 4200 TapeStation System (Agilent Technologies) with TapeStation Analysis Software 5.1. For the Genomic DNA ScreenTape, 1 μL of the extracted sample was mixed with the sample buffer, and a 100 bp lower marker was added in each lane. The Genomic DNA ScreenTape has an upper limit for sizing DNA fragments of up to 60 kbp. For the High Sensitivity DNA ScreenTape, 2 μL of the extracted sample was mixed with the sample buffer, with each lane containing a 15 bp lower marker and a 10 kbp upper marker. The High Sensitivity DNA ScreenTape has an upper limit for sizing DNA fragments of 5 kbp.

Human DNA samples were analyzed using the Femto Pulse system (Agilent Technologies). Samples were diluted to a concentration range of 50-500 pg/μL, and 2 μL of each diluted sample was loaded into the system. The Femto Pulse system provides high-resolution analysis with an upper limit for sizing DNA fragments of 165 kbp.

### ii. Polyelectrolyte Migration

The mechanism for electrohydrodynamic migration has been the subject of a number of theoretical (5, 6), numerical (7-11), and experimental investigations (1, 4, 12, 13). Here we derive a coarse-grained model combining kinetic theory for the end-to-end vector (14-16) with a freely-jointed chain (FJC) for the internal degrees of freedom (6, 10). Here we used the simplest kinetic theory, based on the FENE-P model, and neglecting hydrodynamic interactions (15, 16, 22). The ratio of the transverse and axial mobilities is shown in FIG. 7A as a function of Weissenberg number Wi.

To compare with experimental measurements (4), parameters representative of λ-DNA: Kuhn step I_{K}=0.1 μm, contour length L=16 μm, and relaxation time λ=0.1 s (23) were chosen. The experiment had a centerline fluid velocity of 1.2 mm/s in a 100 μm channel, leading to a Weissenberg number Wi=2.4. The gives the migration velocity from FIG. 7A as 0.16% of the electrophoretic velocity (50 μm/s) or 0.08 μm/s. The diffusion coefficient of λ-DNA is 0.5 μm²/s (24), giving a characteristic thickness of 6 μm. Observations using confocal microscopy (FIG. 7B) (4) confirm the scale of the concentration layer near the wall (FIG. 7B). Note that in the current experiments the fields are several times larger, and it is likely that steric repulsion is the limiting factor setting the thickness of the high-concentration layer.

FIG. 7C shows the distribution of DNA in the region behind the separation channel. The DNA is distributed in a thin uniform sheet, with a width about the same as the separation channel (80 μm). The rapid drop in the shear and electric fields as DNA exits the separation channel means that it is convected upstream by a weaker electrophoresis until it diffuses back to the channel center, where the larger fluid velocity convects the DNA downstream once more.

### iii. Mass Balance

Over various device configurations (1, 12) and experimental conditions, there always seems to be an upper limit to how much DNA can be trapped, in the range of 2-5 ng/μL. This can be understood as a balance between the trapping mechanism, which constantly feeds DNA into the trap by recirculation from the separation channel (FIG. 1A), and diffusion, which transports DNA from the walls of the device towards the center, where it reenters the flow stream. To determine where the DNA is being lost, mass balances were made, comparing amounts of DNA injected with the amount trapped. Fluorescence measurements towards the outlet end of the separation channel were used to determine the amount of DNA exiting the channel after the flow is turned off and the electric field is reversed. The average concentration near the channel outlet (based on the fluorescence intensity compared with standard solutions) is shown in FIG. 8A. There is a peak in the concentration at the outlet after the field is turned off which corresponds to the electrophoretic velocity (˜1 mm/s). All the DNA is flushed from the channel in less than 5 minutes.

From the velocity of the DNA during flushing (based on the measured electric field in the channel) and the average concentration (FIG. 8A), the amount of DNA that was trapped in the device can be estimated by integrating the concentration over time. The results for two different injected amounts are shown in FIG. 8B. In the 20 mm channel (blue), about 25% of the injected DNA is trapped, regardless of the amount injected. DNA extracted from similar experiments (FIG. 5) indicated maximum yields of 12-15%, implying that about half the DNA is lost during extraction. Using longer channels is a simple way to increase the yield of the device; in the 80 mm channel the trapping is doubled to about 50% of the injected amount, implying an extracted yield of about 25%. The yields from the E. coli extractions were smaller; up to 8% of the injected amount (Table 1).

### iv. Trapping Human DNA

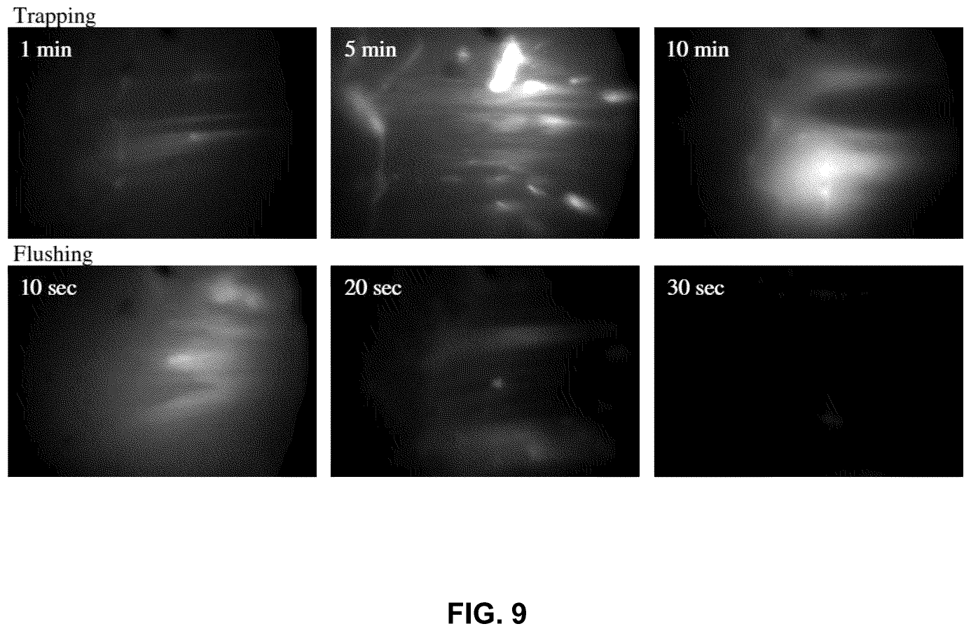

FIG. 9 shows images of human DNA trapped near the entrance to an 80 mm long channel (FIG. 6). Similar to E. coli DNA (FIG. 2), the concentration builds over times of 10 minutes and dissipates in about 30 seconds. Ideally, a larger channel (>0.5 mm) would be used to accommodate the size of human DNA, but the present configuration is functional if not optimal.

The concentrations of the three extracted samples (10 μL each) were 5.1 ng, 1.4 ng, and 4.3 ng. Electropherograms of the two more concentrated extracts (4.3 and 5.1 ng/μL) were similar to the results for E. coli DNA, while the dilute sample shows only a weak peak at 60 kbp. The low concentration of Extract 2 is most likely due to the uncertainties in manual extraction. The lysate and extracts were diluted to the range of the Femto Pulse (50-500 pg/μL) and the electropherograms are shown alongside the TapeStation results in FIGS. 10A1-10B6. The average fragment size in Extracts 1 & 3 is in excess of 50 kbp, with many of them exceeding the maximum read length of the gel (165 kbp). This suggests the method can be used to extract samples for NGS, where the average read length is about 10 kbp (25).

### v. References for the Present Example

1. B E Valley, A D Crowell, J E Butler, A J C Ladd, Electro-hydrodynamic extraction of DNA from mixtures of DNA and bovine serum albumin. Analyst 145, 5532-5538 (2020).
2. P Agrawal, K D Dorfman, Microfluidic long DNA sample preparation from cells. Lab Chip 19, 281-290 (2019).
3. C Lee, S Lee, S G Shin, S Hwang, Real-time PCR determination of rRNA gene copy number: absolute and relative quantification assays with Escherichia coli. Appl. Microbiol. Biotechnol. 78, 371-376 (2008).
4. M Arca, A J C Ladd, J E Butler, Electro-hydrodynamic concentration of genomic length DNA. Soft Matter 12, 6975-6984 (2016).
5. J E Butler, O B Usta, R Kekre, A J C Ladd, Kinetic theory of a confined polymer driven by an external force and pressure-driven flow. Phys. Fluids 19, 113101 (2007).
6. A J C Ladd, Electrophoresis of sheared polyelectrolytes. Mol. Phys. 1, 3121-3133 (2018).
7. O B Usta, J E Butler, A J C Ladd, Transverse migration of a confined polymer driven by an external force. Phys. Rev. Lett. 98, 098301 (2007).
8. W C Liao, et al., Conformation dependence of DNA electrophoretic mobility in a converging channel. Electrophoresis 31, 2813-2821 (2010).
9. R Kekre, Ph.D. thesis (University of Florida) (2011).
10. H Pandey, P T Underhill, Coarse-grained model of conformation-dependent electrophoretic mobility and its influence on DNA dynamics. Phys. Rev. E 92 (2015).
11. D I Kopelevich, S He, R J Montes, J E Butler, Mesoscopic models for electrohydrodynamic interactions of polyelectrolytes. J. Fluid Mech. 915 (2021).
12. R J Montes, J E Butler, A J C Ladd, Trapping DNA with a high throughput microfluidic device. Electrophoresis 40, 437-446 (2019).
13. R J Montes, A J C Ladd, J E Butler, Transverse migration and microfluidic concentration of DNA using Newtonian buffers. Biomicrofluidics 13, 044104 (2019).
14. R B Bird, C F Curtiss, R C Armstrong, O Hassager, Dynamics of polymeric liquids. Volume 2: Kinetic Theory. (John Wiley & Sons, Inc., New York), (1987).
15. M Herrchen, H C Öttinger, A detailed comparison of various FENE dumbbell models. J. Non-Newtonian Fluid Mech. 68, 17-42 (1997).
16. S Yamani, G H Mckinley, Master curves for FENE-P fluids in steady shear flow. J. Non-Newtonian Fluid Mech. 313, 104944 (2023).
17. J D Sherwood, Electrophoresis of rods. J. Chem. Soc., Faraday Trans. 2 78, 1091-1100 (1982).
18. S B Chen, D L Koch, Electrophoresis and sedimentation of charged fibers. J. Colloid Interf. Sci. 180, 466-477 (1996).
19. H Ohshima, Electrophopretic mobility of soft particles. Electrophoresis 16, 1360-1363 (1995).
20. H Ohshima, Henry's function for electrophoresis of a cylindrical colloidal particle. J. Colloid Interface Sci. 180, 299-301 (1996).
21. J G Kirkwood, J Riseman, The intrinsic viscosities and diffusion constants of flexible macromolecules in solution. The J. Chem. Phys. 16, 565-573 (1948).
22. M Davoodi, K Zografos, P J Oliveira, R J Poole, On the similarities between the simplified Phan-Thien-Tanner model and the finitely extensible nonlinear elastic dumbbell (Peterlin closure) model in simple and complex flows. Phys. Fluids 34, 033110 (2022).
23. R E Teixeira, A K Dambal, D H Richter, E S Shaqfeh, S Chu, The individualistic dynamics of entangled DNA in solution. Macromolecules 40, 2461-2476 (2007).
24. R M Robertson, S Laib, D E Smith, Diffusion of isolated DNA molecules: Dependence on length and topology. Proc. Natl. Acad. Sci. U.S.A 103, 7310-7314 (2006).
25. J Dohm, P Peters, N Stralis-Pavese, H Himmelbauer, Benchmarking of long-read correction methods. NAR Genomics Bioinforma. 2 (2020).

### INFORMAL SEQUENCE LISTING

| | | SEQ |
|---|---|---|
| | | ID |
| Description | Sequence | NO: |
| E. coli F primer | GCTACAATGGCGCATACAAA | 1 |
| E. coli R primer | TTCATGGAGTCGAGTTGCAG | 2 |

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A microfluidic trap, comprising:
an inlet region having an injection port, a first extraction port, and a first electrode;
an outlet region having an outlet port and a second electrode; and
a microfluidic channel providing fluidic communication between the inlet region and outlet region.

2. The microfluidic trap of claim 1, wherein the first electrode and the first extraction port are in close proximity and separated from the injection port by an inlet distance.

3. The microfluidic trap of claim 1, wherein the second electrode and the outlet port are separated by an outlet distance.

4. The microfluidic trap of claim 1, wherein the injection port, extraction port, and outlet port are in line with a longitudinal axis of the microfluidic channel.

5. The microfluidic trap of claim 1, wherein the inlet region further comprises one or more reservoirs adjacent to the longitudinal axis of the microfluidic channel, wherein the first electrode is within one of the one or more reservoirs and the second electrode is in line with the longitudinal axis of the microfluidic channel.

6. The microfluidic trap of claim 1, wherein the first electrode and the second electrode comprise a noble metal, one or more metal alloys, or a combination of any thereof.

7. The microfluidic trap of claim 1, wherein the first electrode and second electrode are coated with a charge neutral polymer having a sufficient pore size to prevent passage of genomic DNA or other nucleic acids through the pores.

8. The microfluidic trap of claim 1, wherein the inlet region, outlet region, microfluidic channel, or any combination of any thereof are laser etched in acrylic.

9. The microfluidic trap of claim 1, wherein the inlet region, outlet region, microfluidic channel, or any combination of any thereof comprise glass, silicon, polydimethylsiloxane (PDMS), paper, a thermoplastic material, or a combination of any thereof.

10. A system, comprising:
a microfluidic trap of claim 1;
a voltage generator;
one or more fluid flow valves; and
a fluid flow generator.

11. The system of claim 10, further comprising:
a fluid source in fluidic communication with the inlet region of the microfluidic trap.

12. The system of claim 10, further comprising:
a fluid collection device in fluidic communication with the outlet region of the microfluidic trap.

13. The system of claim 10, further comprising:
a flow valve in fluidic communication with the fluid source.

14. The system of claim 10, wherein the fluid flow generator is an active or passive fluid flow generator.

15. The system of claim 14, wherein the active fluid flow generator is a fluidic pump.

16. The system of claim 14, wherein the passive fluid flow generator is gravitational flow created by a difference in height between the fluid source and the fluid collection device.

17. A method, comprising:
establishing a fluid flow in the system of claim 10;
providing a first electric field across the longitudinal axis of the microfluid channel with the voltage generator;
injecting a biological sample comprising genomic DNA into the injection port of the system;
waiting a first period of time;
stopping the fluid flow;
removing the first electric field;
providing a second electric field across the first and second electrodes of the microfluidic trap with the voltage generator;
waiting a second period of time;
collecting the remaining nucleic acids from the extraction port of the microfluidic trap.

18. The method of claim 17, wherein the biological sample comprises cell lysate comprising genomic DNA.

19. The method of claim 17, wherein the polarity of the first electric field is such that the polarity attracts a polyelectrolyte or nucleotide to the inlet region.

20. The method of claim 17, wherein the polarity of the second electric field is such that the polarity attracts a polyelectrolyte or nucleotide to the inlet region.


SEQUENCE LISTING (2 sequences)

SEQ ID NO: 1 - DNA, 20 bp, synthetic construct
  source 1..20: mol_type = other DNA
gctacaatggcgcatacaaa

SEQ ID NO: 2 - DNA, 20 bp, synthetic construct
  source 1..20: mol_type = other DNA
ttcatggagtcgagttgcag